(19) United States
(12) Patent Application Publication
(10) Pub. No.: US 2014/0214069 A1
(43) Pub. Date: Jul. 31, 2014

(54) Inflatable Embolic Deflector

(71) Applicant: Edwards Lifesciences Corporation, Irvine, CA (US)

(72) Inventor: Michael D. Franklin, Costa Mesa, CA (US)

(73) Assignee: Edwards Lifesciences Corporation, Irvine, CA (US)

(21) Appl. No.: 14/150,566

(22) Filed: Jan. 8, 2014

Related U.S. Application Data
(60) Provisional application No. 61/758,700, filed on Jan. 30, 2013.

(51) Int. Cl. A61F 2/01

(52) U.S. Cl. CPC A61F 2/013
USPC 606/194

(57) ABSTRACT

The present disclosure concerns embodiments of an expandable and collapsible embolic deflector that can be collapsed to a very small profile for insertion through a patient's vasculature and then expanded once inside the body. The embolic deflector comprises a flexible, inflatable frame that supports a blood-permeable membrane that prevents emboli above a predetermined size from entering vasculature that is blocked by the membrane. The frame comprises an inflatable body made of a soft polymeric material, such as urethane, Pebax, or nylon, similar to a balloon of a balloon catheter. In particular embodiments, the frame does not include any metal components. In certain embodiments, the frame can include one or more radiopaque markers, which can be made of a metal or metal alloy. In either case, the risk of potential trauma to the patient can be reduced since the amount of hard metal components is significantly reduced or completely eliminated.

### CROSS REFERENCE TO RELATED APPLICATION

This application claims the benefit of U.S. Provisional Patent Application No. 61/758,700, filed Jan. 30, 2013, which is hereby incorporated by reference.

### FIELD

The present disclosure concerns embodiments of a device for deflecting emboli within a patient's circulation and related methods.

### BACKGROUND

Embolic deflectors are used to divert emboli from entering selected vasculature (e.g., the cerebral vasculature) during surgical procedures, such as heart valve replacement procedures. In some cases, embolic deflectors are configured to be collapsible to a relatively small profile for delivery through a patient's vasculature and expandable to their functional size once inside the body. Such expandable embolic deflectors typically incorporate an expandable and collapsible metal frame, which typically is made of a self-expanding metal such as Nitinol. When the metal frame is expanded inside a lumen of the patient's vasculature (e.g., within the aortic arch), the frame can contact and potentially cause trauma to the inner wall of the lumen.

### SUMMARY

The present disclosure concerns embodiments of an expandable and collapsible embolic deflector that can be collapsed to a very small profile for insertion through a patient's vasculature and then expanded once inside the body. The embolic deflector comprises a flexible, inflatable frame that supports a blood-permeable membrane that prevents emboli above a predetermined size from entering vasculature that is blocked by the membrane. The frame comprises an inflatable body made of a soft polymeric material, such as urethane, Pebax, or nylon, similar to a balloon of a balloon catheter. In particular embodiments, the frame does not include any metal components. In certain embodiments, the frame can include one or more radiopaque markers, which can be made of a metal or metal alloy. In either case, the risk of potential trauma to the patient can be reduced since the amount of hard metal components is significantly reduced or completely eliminated.

In one representative embodiment, an inflatable embolic deflector comprises an elongate shaft having a proximal end and a distal end, and an inflation fluid lumen extending therethrough. A flexible inflatable frame, defining at least one opening, is coupled to the distal end of the shaft. The flexible frame comprises a lumen in fluid communication with the inflation fluid lumen. The frame is configured to inflate when a pressurized inflation fluid is introduced through the inflation fluid lumen and into the lumen of the frame. A membrane is disposed in and spans the at least one opening. The membrane has a permeability such that blood can pass through but emboli greater than a predetermined size cannot pass through.

In another representative embodiment, a method for deflecting emboli within a patient's vasculature comprises inserting an inflatable embolic deflector into the patient's vasculature. The inflatable embolic deflector comprises a shaft, an inflatable frame coupled to the distal end of the shaft, and a membrane supported by the frame. The membrane has a permeability such that blood can pass through but emboli greater than a predetermined size cannot pass through. The frame is in a deflated configuration when the deflector is introduced into the patient's vasculature. Once inside the patient's vasculature, the frame is inflated and then situated to cover an ostium of a lumen of the patient's vasculature such that that blood can pass through the membrane and into the lumen but emboli greater than the predetermined size cannot.

The foregoing and other objects, features, and advantages of the invention will become more apparent from the following detailed description, which proceeds with reference to the accompanying figures.

### BRIEF DESCRIPTION OF THE DRAWINGS

FIG. 1 is a perspective view, FIG. 2 is a side view, FIG. 3A is a proximal view, and FIG. 3B is a distal view of one embodiment of an embolic deflector.

FIGS. 4-5 illustrate another embodiment of an embolic deflector.

FIG. 6 illustrates one embodiment of a membrane portion of an embolic deflector.

FIG. 7 illustrates brachial artery insertion of an embolic deflector, according to one embodiment.

FIG. 8 illustrates femoral artery insertion of an embolic deflector, according to one embodiment.

FIGS. 9A-9C illustrate a method of deployment of an embolic deflector through a patient's right arm, according to one embodiment.

FIGS. 10A-10C illustrate an alternative method of deployment of an embolic deflector through a patient's femoral artery.

FIG. 11 illustrates an embodiment where an embolic deflector is used in combination with a filter spanning the aortic lumen and placed downstream of the left subclavian artery but upstream of the renal arteries.

FIG. 12 illustrates an exemplary embodiment of a delivery apparatus comprising an embolic deflector and a delivery sheath.

FIG. 13 illustrates one embodiment of a torque control mechanism for an embolic deflector.

FIG. 14 illustrates components of one embodiment of an embolic deflector deployment kit.

FIGS. 15A-15K illustrate various embodiments of embolic deflectors in end view.

FIGS. 16A-16L illustrate various embodiments of embolic deflectors in side view.

FIGS. 17A-17D illustrate various embodiments of a locking mechanism between an embolic deflector shaft and an introducer sheath.

### DETAILED DESCRIPTION

Disclosed herein are inflatable embolic deflection systems that include a deflector, along with associated deployment and removal systems, that can advantageously prevent emboli above a predetermined threshold size from entering the cerebral vasculature that may be dislodged, such as during a surgical procedure, such as a procedure for repairing a native heart valve or replacing a native heart valve with a prosthetic valve. As such, the risk of potentially life-threatening transient ischemic attacks or embolic strokes can be reduced or eliminated. Conventional embolic filters are primarily configured to capture, retain and retrieve embolic material. In contrast, deflectors as disclosed herein in particular embodiments are configured to deflect or otherwise divert embolic material to a location downstream (relative to the direction of blood flow in the vessel in which the deflector is deployed) of the deployed location of the deflector to a less critical region of the body rather than the brain and other tissues perfused by the carotid and vertebral arteries. Once downstream, the emboli can be acted upon by physiologic anticoagulation mechanisms and/or externally administered anticoagulants. When in use, the emboli need not necessarily physically come into contact the embolic deflection device for the device to be effective, so long as the emboli are prevented from travelling through the deflector and are instead diverted downstream as noted above. In some embodiments, the deflector can be deployed in the aortic arch over the ostia of the brachiocephalic and the left common carotid arteries. In some embodiments, the deflector can be deployed in the aortic arch over the ostia of the brachiocephalic, left common carotid, and the left subclavian arteries. The right common carotid artery and the right subclavian artery normally branch off the brachiocephalic artery. The right vertebral artery normally branches off the right subclavian artery, while the left vertebral artery normally branches off the left subclavian artery. While the deflector is configured to deflect emboli greater than a pre-determined size, such as 100 microns for example, into the descending aorta, the deflector is also preferably configured to be sufficiently porous to allow adequate blood flow through the ostia of the vessels which the deflector may contact, such as the brachiocephalic, left common carotid, and/or left subclavian arteries, so as to sufficiently maintain perfusion to the brain and other vital structures.

The method advantageously allows for deflection of emboli flowing within a main vessel, such as the aorta, from entering a side branch vessel, such as the brachiocephalic artery, left common carotid artery, and/or left subclavian artery while allowing deflection of the emboli further downstream in the main vessel (e.g., the aorta) perfusing less critical body organs and other structures, and allowing for lysis of the emboli via physiologic and/or pharmacologic declotting mechanisms. A side branch vessel as defined herein is a non-terminal branch vessel off a main vessel, such that the main vessel continues proximally and distally beyond the ostia of the side branch vessels. For example, the brachiocephalic artery, left common carotid artery, and left subclavian arteries are side branch vessels of the aorta, which continues distally toward the abdomen past the ostia of the aforementioned side branch vessels. This is in contrast to main vessels that can split (e.g., bifurcate) into terminal branch vessels such that the main vessel no longer exists distal to the ostia of the terminal branch vessels. One example of a main vessel that splits into terminal branch vessels is the abdominal aorta, which terminates distally subsequent to its bifurcation into the common iliac arteries.

In some embodiments, the deflector can be placed in a first axial, collapsed orientation through a first insertion site, such as an artery of an upper extremity, that is distinct from a second insertion site, such as a femoral or contralateral upper extremity, for catheters and other devices used for an index procedure. In some embodiments, the embolic deflector can be deployed with no greater than about a 6 French sheath, and can be readily placed using a standard Seldinger technique. The device can be collapsed into its reduced crossing profile orientation through a loader, backloaded past the hemostasis valve of a sheath, and then advanced through the sheath into a first branch vessel, such as the brachiocephalic artery, and then into a main vessel, such as the aorta. Within the aorta, the deflector can be expanded into an expanded transverse orientation once removed from the sheath, and is positioned across the ostia of one or more branch vessels to deflect emboli downstream (with respect to the direction of blood flow in the aorta) into the descending aorta.

In the expanded configuration, the deflector generally has a major axis with a length that is greater than the length along a transverse, or minor axis. As deployed within the vessel, the major axis is generally aligned in the direction of blood flow, such that a first end of the deflector residing on the major axis points in an upstream direction and a second, opposing end of the deflector also residing on the major axis points in a downstream blood flow direction.

A first end of the deflector can thus be aligned or permitted to self-align and can be secured in position extending upstream in the aorta covering, for example, the ostia of a branch vessel, such as the innominate artery. The deflector can also be configured to simultaneously have a second end extending downstream in the aorta to cover the ostia of a second branch vessel (e.g., the left common carotid artery).

The embolic deflector is able to be placed before an index procedure is begun and can remain in place, providing embolic deflection, until the procedure is completed, or for a shorter or longer period of time as clinically indicated. In some embodiments, the deflector has a very low profile in the aorta so that wires, catheters, and sheaths can pass by it without interference. In some embodiments, the deflector is configured to deflect emboli greater than, for example, 100 microns in size away from the carotid arteries thus protecting the patient from potentially devastating neurological consequences of these emboli. The deflector can be designed so that one size fits all, or may be provided in a series of graduated sizes.

In some embodiments, a method of reducing the risk of emboli entering the cerebral circulation as a consequence of an index procedure in the heart or another blood vessel, such as the aorta, involves the following steps. First, an elongate, flexible shaft is inserted into the vasculature at a point other than a femoral artery, or in some embodiments a contralateral femoral artery from that of the insertion point for the index procedure. A deflector carried on the shaft is then positioned in the aorta such that it spans the ostium of one, two, or more of the brachiocephalic, left common carotid, and left subclavian arteries. An index procedure catheter is then introduced into a femoral artery. The index procedure catheter is then advanced across the thoracic aorta to a treatment site in the heart or a blood vessel. The index procedure is then performed. The index procedure catheter can also be inserted into the body via an entry point in a vessel other than a femoral artery, via a transapical approach, or via a transaortic approach. Some non-limiting examples of index procedures include valve replacement procedures, including aortic and mitral valve replacement, including transcatheter aortic or mitral valve implantation, aortic or mitral valvuloplasty, including balloon valvuloplasty, heart valve repair, coronary angioplasty, or coronary artery bypass grafting. Following completion of the index procedure, the index procedure catheter is removed from the patient. The deflector is then removed from the patient.

In other embodiments, the method includes introducing an elongate, flexible shaft into the aorta, such as via the brachiocephalic artery, the shaft carrying a deflector thereon. The deflector is then positioned in the aorta such that it spans the ostium of one, two, or more of the brachiocephalic, left common carotid, and left subclavian arteries. An index procedure is then performed on the heart or other vessel, such as the aorta. The deflector can then be removed from the patient. The index procedure could be performed via open surgical access, a less invasive thoracoscopic approach, transapically, percutaneously, or even noninvasively (e.g., an external DC cardioversion) in some embodiments.

One embodiment of a method of using an embolic deflector to reduce the risk of emboli from entering the circulation during a Balloon Aortic Valvuloplasty (BAV) procedure will now be described. Wire access is gained through any appropriate access, such as the right radial or brachial artery and advanced to the ostium of the brachiocephalic artery. A 6 French Sheath with a dilator is then inserted over the wire. The sheath tip is positioned at the ostium of the brachiocephalic artery. An embolic deflector is inserted into the sheath and deployed in the aorta. The device positioning can be confirmed with fluoroscopic imaging. A BAV catheter is inserted into the body, such as via the femoral artery. A Balloon Aortic Valvuloplasty catheter is advanced into the descending aorta, around the aortic arch passing by the deflector. The BAV catheter is then positioned across the aortic valve. The balloon is inflated and deflated against the stenotic and calcified aortic valve. The BAV catheter is then removed, passing by the embolic deflector during the retrieval process, through the femoral artery access site. The embolic deflector and sheath are removed from the radial or brachial artery.

One embodiment of a method of using an embolic deflector to reduce the risk of emboli from entering the circulation during a Transcatheter Aortic Valve Implantation (TAVI) will now be described. Wire access is gained through the right radial or brachial artery and advanced to the ostium of the brachiocephalic artery. A 6 French Sheath with a dilator is then inserted over the wire. The sheath tip is positioned at the ostium of the brachiocephalic artery. A deflector is inserted into the sheath and deployed in the aorta. The device positioning can be confirmed with fluoroscopic imaging. Multiple wires and catheters can then be used to assess the aortic valve and arch anatomy and to dilate the native aortic valve prior to the deployment of the transcatheter aortic valve. These devices can be inserted via the femoral artery and pass the deflector. The transcatheter aortic valve is then inserted via a delivery system or catheter which can be inserted via the femoral artery. A transapical, trans-septal, or transaortic approach could be employed in some embodiments. The TAVI catheter is advanced into the descending aorta, around the aortic arch passing by the deflector. The transcatheter aortic valve is then positioned and deployed in the native aortic valve. The TAVI catheter is then removed, passing by the deflector device during retrieval through the femoral access site. Once the TAVI catheter is removed, the deflector device and sheath are removed from the radial or brachial artery. Further details of replacement valves and methods of valve implantation that can be used with the deflectors described herein can be found, for example, in U.S. Pat. No. 7,618,446 to Andersen et al., U.S. Pub. No. 2008/0004688 to Spenser et al., U.S. Pat. Pub. No. 2007/0043435 to Seguin et al., U.S. Pat. Pub. No. 2008/0140189 to Nguyen et al., U.S. Pat. No. 8,187,299 to Goldfarb et al., and U.S. Pat. No. 8,216,301 to Bonhoeffer et al., all of which are hereby incorporated by reference in their entireties.

Deployment of a deflector as described herein can be advantageous for a variety of applications. The applications may include use during a wide range of operative procedures, including but not limited to open cardiothoracic, mediastinoscopy, transapical, or percutaneous procedures. For example, the embolic deflector could be deployed prior to an angioplasty procedure, such as a balloon angioplasty or rotational atherectomy involving one, two, or more coronary arteries. The deflector could also be deployed prior to a heart valve procedure, such as an open, transapical, or percutaneous mitral or aortic valve replacement or repair or valvuloplasty procedure. In some embodiments, the deflector could be deployed prior to repair of an aortic aneurysm and/or dissection. In still other embodiments, the deflector could be deployed prior to electrical or pharmacologic cardioversion of an arrhythmia where there may be an increased potential risk of embolization following return to normal sinus rhythm post-cardioversion, such as in atrial fibrillation, atrial flutter, multifocal atrial tachycardia, ventricular tachycardia, ventricular fibrillation, or torsades de pointes for example. In some embodiments, the embolic deflector could be utilized in any index procedure involving the passage of catheters crossing the atrial septum, including cardiac ablation procedures of ectopic atrial or ventricular foci, leading to arrhythmias. Other examples of index procedures could include repair of shunt defects, including atrial septal defects, ventricular septal defects, patent foramen ovale, and Tetralogy of Fallot.

In some embodiments, the deflector is deployed within a patient no more than about 48 hours, 36 hours, 24 hours, 12 hours, 8 hours, 6 hours, 4 hours, 3 hours, 2 hours, 1 hour, or less prior to the index procedure. In some embodiments, the deflector is removed from a patient no sooner than 1, 2, 3, 4, 5, 10, 15, 20, 30, 40, 50, 60 minutes, or more following completion of the index procedure.

In some embodiments, deflectors as disclosed herein can be deployed into the venous circulation, such as in the superior or inferior vena cava, for the prevention of pulmonary embolism.

In some embodiments, the deflector can be deployed for short-term or long-term protection against emboli even when an operative procedure may not be contemplated, such as, for example, with a hypercoaguable state, cancer, atrial fibrillation, endocarditis, rheumatic heart disease, sepsis, including fungal sepsis, patent foramen ovale, atrial septal defect, ventricular septal defect, other arteriovenous shunt, or patients already having an implanted prosthetic device prone to emboli formation, such as having a prosthetic heart, left ventricular assist device, replacement mitral or aortic valve, and the like. For example, a patient may be on anticoagulant therapy for one, two, or more of the aforementioned conditions, but need to temporarily discontinue the medication for an upcoming procedure, or the medication may be temporarily contraindicated because of an acute bleed such as a gastrointestinal bleed, and thus be at risk for embolic stroke. A deflector can thus be deployed for the period of time in which the patient has discontinued their anticoagulation therapy, which may be more than about 12, 18, 24, 36, 48, 72 hours, or more. In other embodiments, the deflector can be configured for more long-test implantation, such as for at least about 1, 4, 6 or 8 weeks, or even more. However, in other more short-term applications, the deflector is deployed within the body for no more than about 24, 18, 12, 6, 4, 3, 2, 1 hour, or even less.

In some embodiments, the device may also be deployed into a position in which one edge is inside the brachiocephalic artery, covering the ostium of the right common carotid, and in which the opposite edge extends into the aortic lumen and covers the ostium of the left common carotid artery, leaving the brachiocephalic ostium substantially unobstructed by the deflector.

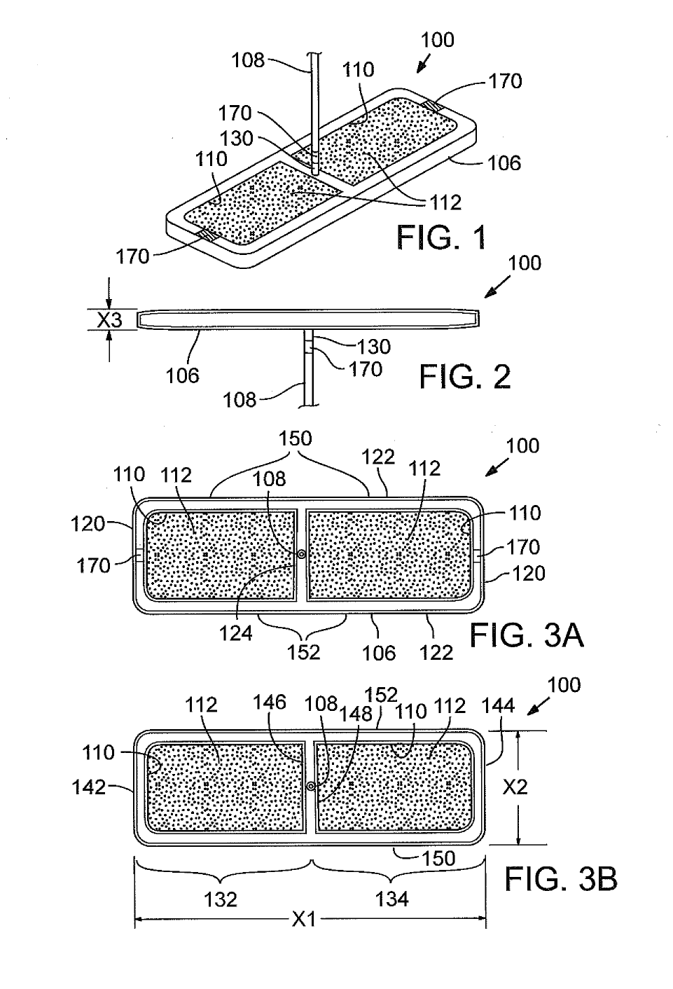

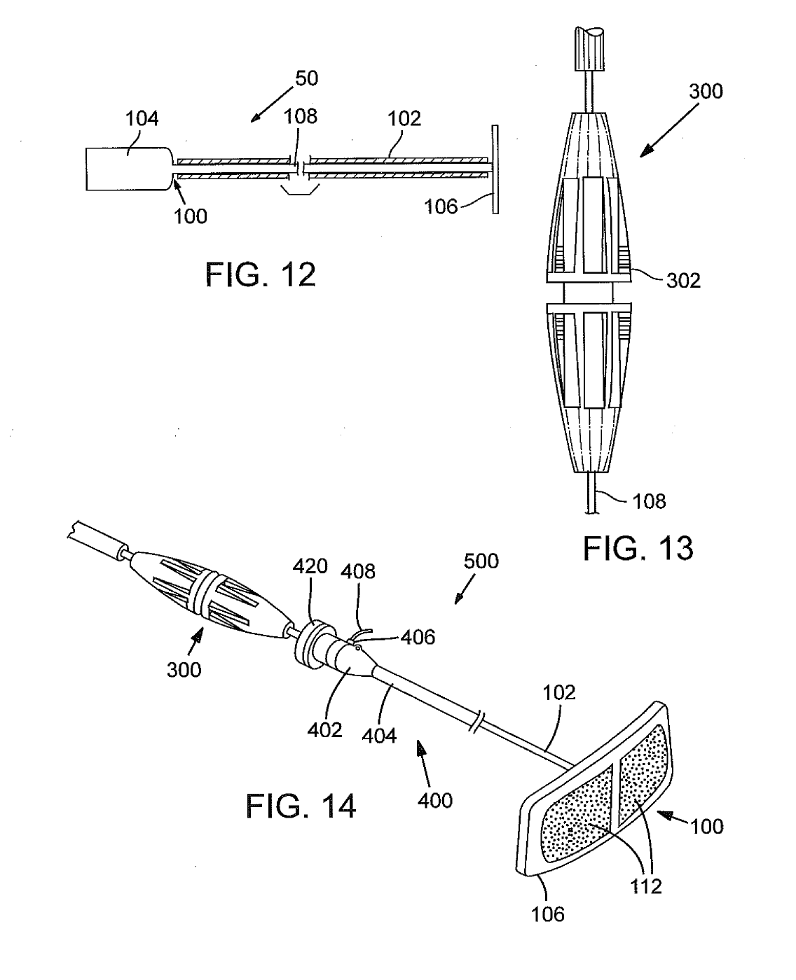

FIGS. 1-3B illustrate an embolic deflector 100 in accordance with one embodiment. As shown in FIG. 12, the deflector 100 can be a component of an embolic deflector delivery system 50, which can also include an elongated delivery sheath 102. The deflector 100 can comprise a handle 104 at the proximal end of the device, a flexible, inflatable frame 106 at the distal end of the device, and an elongated, flexible shaft 108 extending between and connected to the handle 104 and the frame 106. The shaft 108 extends axially through the sheath 102 and both components desirably are moveable longitudinally and rotationally relative to each other. In certain embodiments, the sheath 102 and the deflector 100 are separate components that can be introduced into a patient's vasculature sequentially as further described below. In other embodiments, the sheath 102 and the deflector 100 can be part of the same delivery apparatus that are introduced together into a patient's vasculature. In such embodiments, the proximal end of the sheath 102 can be coupled to the handle 104, which can have one or more actuators to effect longitudinal and/or rotational movement of the sheath 102 relative to the shaft 108, and/or longitudinal and/or rotational movement of shaft 108 relative to the sheath 102.

Referring to FIGS. 1-3B, the frame 106 in the illustrated embodiment defines two openings 110, each of which is spanned by a respective membrane 112. The deflector 100 can also be provided with one or more radiopaque markers 170 (three are shown in the illustrated embodiment) and a frame attachment junction 130. FIG. 2 illustrates a side view of the flexible frame 106, the shaft 108, the junction 130 and one radiopaque marker 170. As best shown in FIGS. 3A-3B, (illustrating proximal and distal surfaces of the frame 106, respectively) the frame 106 comprises two peripheral end struts 120, two peripheral side struts 122 extending between respective ends of the end struts 120, a central strut 124 extending from one side strut 122 to the other side strut 122, and two membranes 112, each spanning one of the openings 110 defined by the frame 106.

The frame 106 in particular embodiments has a thin wall construction similar to a balloon of a balloon catheter. As such, the framer 106 has a lumen for receiving an inflation fluid that extends through the end struts 120, the side struts 122 and the central strut 124. The shaft 108 includes a lumen that is in fluid communication with the lumen of the frame 106 and in fluid communication with a source of an inflation fluid (e.g., saline) at the proximal end of the shaft 108. For example, the handle 104 (FIG. 12) can have a port that is fluidly connectable to a source of an inflation fluid. The handle 104 can have a respective lumen that receives an inflation fluid from a source and directs the fluid to flow into the lumen of the shaft 108. In a delivery configuration, the frame 106 is deflated and can be folded in half against the shaft 108 for delivery into a patient's vasculature. The two folded halves of the deflated frame 106 can be wrapped around the shaft 108 to reduce the profile of the frame as much as possible for introduction into the body. When the frame 106 is positioned at the desired deployment location within the body (e.g., within the aortic arch), a pressurized inflation fluid is introduced into the frame 106 via the shaft 108 to cause the frame 106 to inflate and assume the overall shape shown in FIGS. 1-3B.

In this embodiment, the internal pressure provides the flexible frame 106 with structural rigidity inside the patient's circulation. In general, the introduction of more fluid will create greater internal pressure and thus greater structural rigidity. In this embodiment, the inflated flexible frame 106 can then be positioned to cover the ostia of one or more lumen in the patient's circulation while an index procedure is completed, such that blood can flow through the membranes 112, but emboli are deflected downstream. When the deflector 100 is required to be removed from the patient's circulation (e.g., after the index procedure is completed), the inflation fluid can be withdrawn from the frame 106, reducing its volume and internal pressure. Thus the flexible frame 106 can be reduced to a more compact configuration to facilitate its removal from the patient's circulation.

Referring to FIGS. 3A and 3B, the shaft 108 can be attached to the frame 106 near the center point of the central strut 124. The frame 106 can define at least a first lobe 132 and a second lobe 134 formed by the struts 120, 122, 124 and biased in opposing directions. As illustrated, struts 120, 124 can follow a minor axis X2 of the frame, while the struts 122 extend along a major axis X1 of the frame. In some embodiments additional struts may optionally be provided.

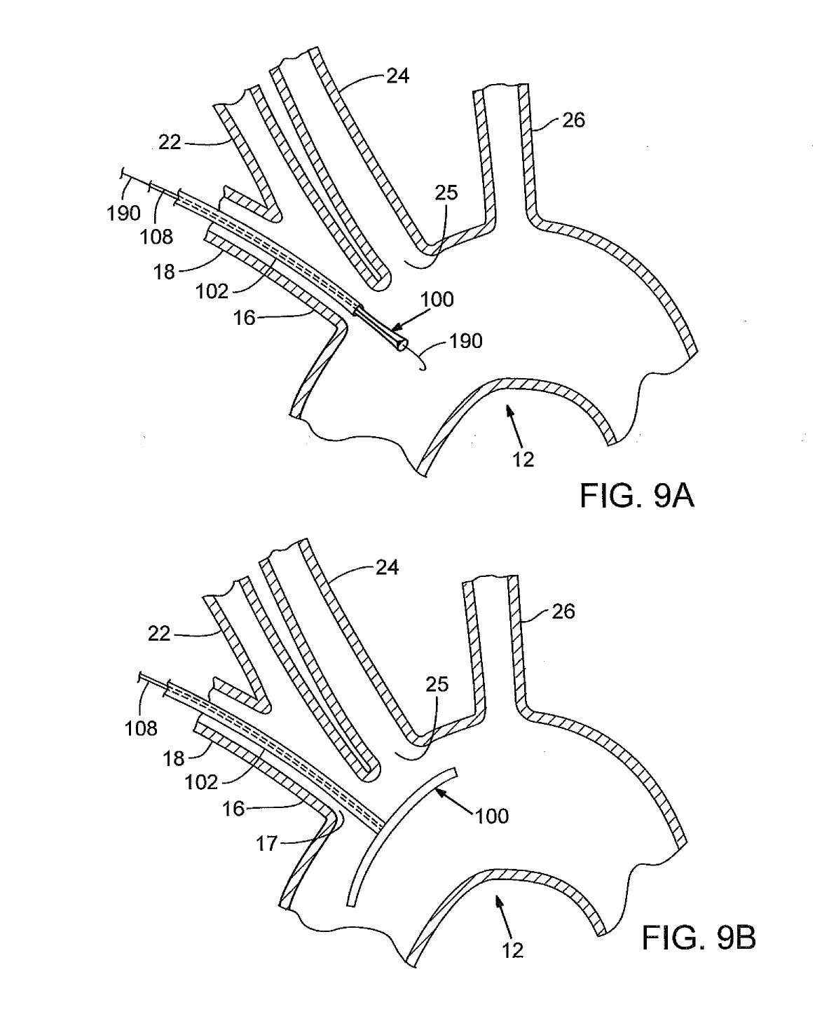

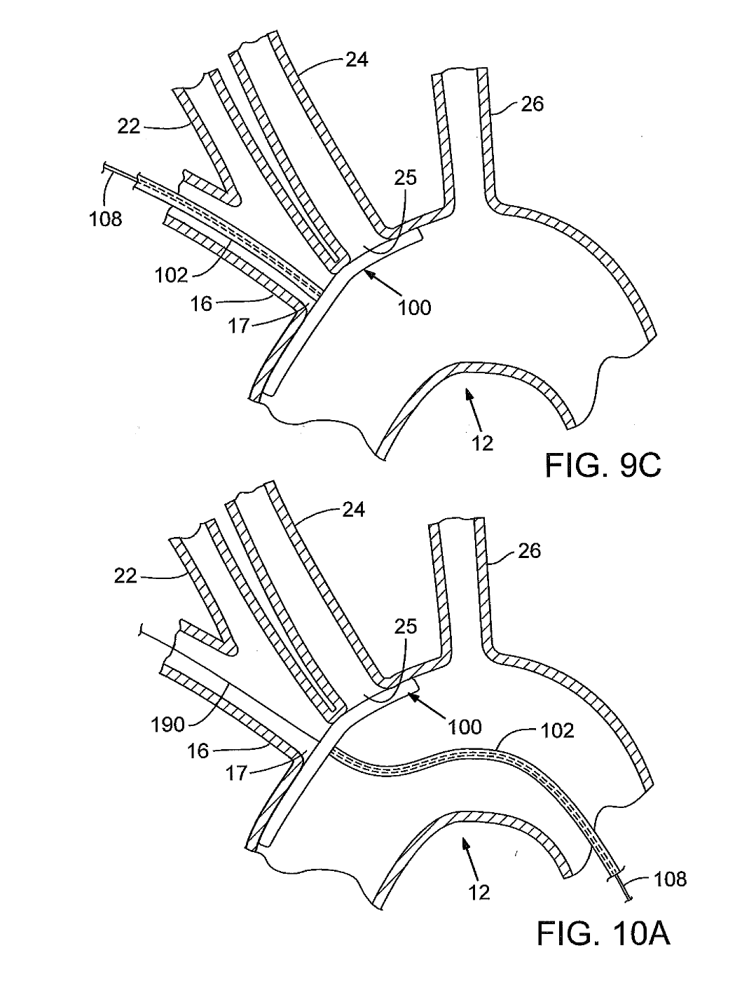

The first lobe 132 has a lateral end 142 and a medial end 146, while the second lobe 134 has a lateral end 144 and a medial end 148. The lobes 132, 134 also have a first side 150 and a second side 152, the distance between sides 150, 152 defining the width X2 of the frame 106. The lobes 132, 134 are movable between an axial orientation prior to delivery (illustrated in FIG. 9A) to a transverse orientation following deployment in the vessel (illustrated in FIG. 9C). In the illustrated embodiment as well as others, the deflector 100 is convertible between a deflated configuration in which both the first end (i.e., the lateral end 142) and the second end (i.e., the lateral end 144) are folded against each other and point in the proximal direction, and a deployed configuration in which the first and second ends 142, 144 point in lateral directions.

Still referring to FIG. 3B, the first lobe 132 is symmetric to, and encloses a surface area that is the same or substantially the same as a surface area enclosed by the second lobe 134. In other embodiments, the first lobe 132 is asymmetric to, and can enclose a surface area that is at least 10%, 20%, 30%, 40%, 50%, 75%, 100% greater, or more than, the surface area enclosed by the second lobe 134. In some embodiments, the frame 106 includes 3, 4, 5, 6, 7, 8, or more lobes projecting radially outwardly from a central hub depending on the patient's particular anatomy and luminal sites to be protected by the deflector 100. In some embodiments, the deflector has only a single plane of symmetry, within which the junction 130 lies (e.g., the plane of symmetry runs coaxial with the shaft 108 and extends across the minor (transverse) axis of the deflector 100).

The frame 106 can be defined as having a major axis (maximum length) X1 between a first lateral end 142 and a second lateral end 144, and a minor axis (maximum width) X2 between a first side 150 and a second side 152 of the frame 106 when laid flat and fully inflated, as well as a height X3 as illustrated in FIG. 2. When fully inflated, the frame 106 can be sized to ensure coverage of both the brachiocephalic and left common carotid artery over a wide range of anatomies.

In some embodiments, the frame 106 has a major axis distance X1 that is at least about 100%, 110%, 120%, 130%, 140%, 150%, 175%, 200%, 225%, 250%, 275%, 300%, 325%, 350%, 400% or more relative to the minor axis distance X2. However, in other embodiments, the distances X1 and X2 are the same or substantially the same.

In some embodiments, the frame 106 has a length X1 of from about 40 mm to about 80 mm, such as from about 50 mm to about 70 mm, such as between about 56 mm to about 60 mm. The frame 106 can have a width X2 of from about 20 mm to about 30 mm, or from about 23 mm to about 27 mm in some embodiments. The frame 106 has a height X3 of from about 7 mm to about 11 mm, such as from about 8.5 mm to about 9.5 mm in some embodiments.

In the illustrated embodiments, the flexible frame 106 may comprise any one of a variety of materials such as nylon, Pebax, urethane, or other thin, soft, polymer film. The frame 106 may be fabricated in some embodiments by stamping multiple layers of the material into the desired shapes and heat sealing them together.

In the illustrated embodiments, the shaft 108 can be an elongate, flexible hollow tube that can be made of a variety of materials, examples of which are disclosed with respect to the frame 106 materials above. The shaft 108 can be designed to have flexibility, column strength, and to resist stretching under tension.

The length of the shaft 108 will depend upon the intended vascular access point. In some embodiments, the shaft 108, or the entire deflecting device including the shaft 108, is from about 100 cm to about 120 cm, such as about 110 cm in length to allow for manipulation through sheaths as long as 90 cm, or more. The shaft 108 can have a low profile outer diameter, such as between about 0.030 inches and 0.040 inches, or about 0.035 inches in some embodiments so that the physician can flush contrast between the shaft 108 and the sheath to confirm position of the shield.

One embodiment of the membrane 112 of the deflector 100 is illustrated in FIG. 6. The membrane 112 is configured to have a porous surface or include apertures so as to allow for blood flow sufficient to perfuse the brain and other important structures served by the carotid and vertebral arteries, but also deflects emboli greater than a size which is likely to cause an embolic stroke of clinical significance. In some embodiments, the membrane 112 has pores that are no more than about 200 μm, 175 μm, 150 μm, 125 μm, 100 μm, 75 μm, 50 μm, or even smaller in size. The membrane 112 can be made of any of a variety of biocompatible materials, including, but not limited to polyurethane, Pebax, Nylon, PET, PETE, PETN, PTFE, polypropylene, polyacrylamide, silicone, polymethylmethacrolate, GoreTex™, or ePTFE with a high internodal distance. The wall thickness of the membrane 112 can be about 0.0001-0.005 inches, or about 0.0005-0.0015 inches in some embodiments. The wall thickness may vary depending on the particular material selected. In some embodiments, the pores or other perfusion openings may be laser-drilled out of the membrane material, or a heated rod or other device could be used. The membrane 112 could be either elastic or non-elastic. The membrane 112 may have either uniform or non-uniform pore sizes and areal distributions and patterns. In some embodiments, the membrane 112 can be optionally filled or coated with a radiopaque material, and may be woven, extruded or otherwise film-formed, or airlaid.

In some embodiments, one, two, or more therapeutic agents are operably attached to the membrane 112. The therapeutic agent could include an anticoagulant or clot-dissolving agent, such as, for example, heparin, hirudin, enoxaparin, fondaparinux, abciximab, epitibatide, tirofiban, aspirin, clopidogrel, warfarin, ticlopidine, tissue plasminogen activator, or urokinase. The therapeutic agent could also include an immunosuppressant or antiproliferative agent, such as, e.g., paclitaxel, rapamycin, zotarolimus, prednisone, cyclosporine, methotrexate, mycophenolate, azathioprine, 6 MP, or tacrolimus. Other drugs or bioactive compounds could also be included depending on the desired clinical result.

In some embodiments, the attachment of the membrane 112 to the frame 106 is accomplished by overlapping the membrane 112 about the frame 106 and heat bonding it to a backing membrane, and then trimming the bonded edge, as described hereafter. Other options for attachment include using a polymer, such as a polyurethane dispersion to coat the frame 106 and then utilizing heat bonding, adhesive bonding, suturing, self-wrapping and bonding, mechanical bonding such as an interference fit by a double frame trapping the membrane material around the edges, stitching and/or ultrasonic welding. In some embodiments, a dip process could be used to attach the membrane 112 to the frame 106, similar to that of dipping a wand head into soap for blowing bubbles.

In some embodiments, the radiopaque marker elements 170 are made of a metal or a metal alloy, such as, for example, one or more of Nitinol, Elgiloy™, Phynox™, MP35N, stainless steel, nickel, titanium, gold, rhenium, tungsten, palladium, rhodium, tantalum, silver, ruthenium, and hafnium. The marker element could be a 90% platinum and 10% iridium alloy in one particular embodiment. The radiopaque markers 170 disposed on the frame 106 or other portions of the deflector 100 may be welded, plated to the frame surface, painted thereon, dyed, applied as a wire wrap or coil, or any other suitable technique that allows for radiopaque marking. In some embodiments, the markers 170 may be offset from the major axis of the frame to permit optimal folding of the frame 106. While the markers 170 could be located in any clinically desirable location along the deflector 100, in some embodiments, the frame 106 includes one, two or more markers 170 on or centered about each lateral end 142, 144 as illustrated, as well as one, two, or more markers on the shaft 108 for alignment with a radiopaque marker on the sheath. The radiopaque markers 170 on the frame lateral ends 142, 144 and on the shaft 108 as well as the visibility of the frame 106 itself (if the frame 106 is at least somewhat radiopaque) aid in placement guidance.

In some embodiments the deflector 100 can include a flexible frame 106 having a size sufficient to surround or support a deflection membrane across the ostia of both the brachiocephalic and left common carotid arteries while the deflector 100 is positioned in the aorta, specifically within the aortic arch region of the aorta. However, in other embodiments, the deflector 100 could be sized to cover the ostia of a single vessel, or a first deflector 100 could be sized to cover the ostia of a first vessel, and a second deflector 100 could be sized to cover the ostia of a second vessel. The frame 106 can be flexible, and take a wide variety of shapes to allow continuous or substantially continuous contact with the sidewall of the aortic arch lumen.

While the frame 106 can be substantially flat from a first lateral end to a second lateral end, in some embodiments, the frame 106 is formed so that it has a concave shape when inflated (as shown in FIG. 9B), and in some embodiments the frame 106 can have a compound curvature to form a fitting seal against the aortic wall when it is deployed. In part due to its geometry as described maintaining a concave shape when fully expanded, the deflector 100 advantageously creates a seal along a vessel well, such as the aortic arch, for positioning over the ostia of the brachiocephalic and the left common carotid arteries.

In all of the foregoing illustrations, the deflector 100 is illustrated as it would appear in an unconstrained expansion. In vivo, it is intended that the flexibility of the deflector 100 be sufficient that it can conform (i.e. bend) to the interior wall of the native vessel, under relatively mild proximal traction on the shaft 108, without deforming the configuration of the native vessel. Thus, the periphery of the frame 106 is configured such that along its entire length or at least about 90% of the length of the frame 106 will lie in contact with the inner wall of the vessel.

The aspect ratio of the deflector 100 may be optimized to the intended anatomy in which the deflector is to be used. In one implementation of the invention, the length of the deflector 100 is approximately 2.3 inches and the width is approximately 0.82 inches. The radius of curvature of the ends of the deflector is about 0.41 inches. Thus, the radius of curvature of the ends of the deflector is approximately 50% the width of the deflector. In some embodiments, the radius of curvature of the ends of the deflector is between 25% and 75% of the width of the deflector, more specifically between 40% and 60%, in some embodiments between 45% and 55%, and, in one particular embodiment, between 49% and 51% of the width of the deflector.

In some embodiments, the frame 106 is configured for long-term implantation and embolic protection. As such, the frame 106 may include a plurality of anchors, such as barbs that can be located anywhere along the length of the frame 106, such as at the lateral ends. The shaft in such instances can be detachable from the frame upon implantation. In some embodiments, it may be desirable for the deflector 100 to be either partially or fully biodegradable over a period of time in which the patient may be at a lesser risk for continued embolic formation, such that manual removal of the deflector 100 may advantageously not be necessary. As such, temporary embolic deflector devices could either be configured for manual removal as described elsewhere herein, or biodegradable in other embodiments.

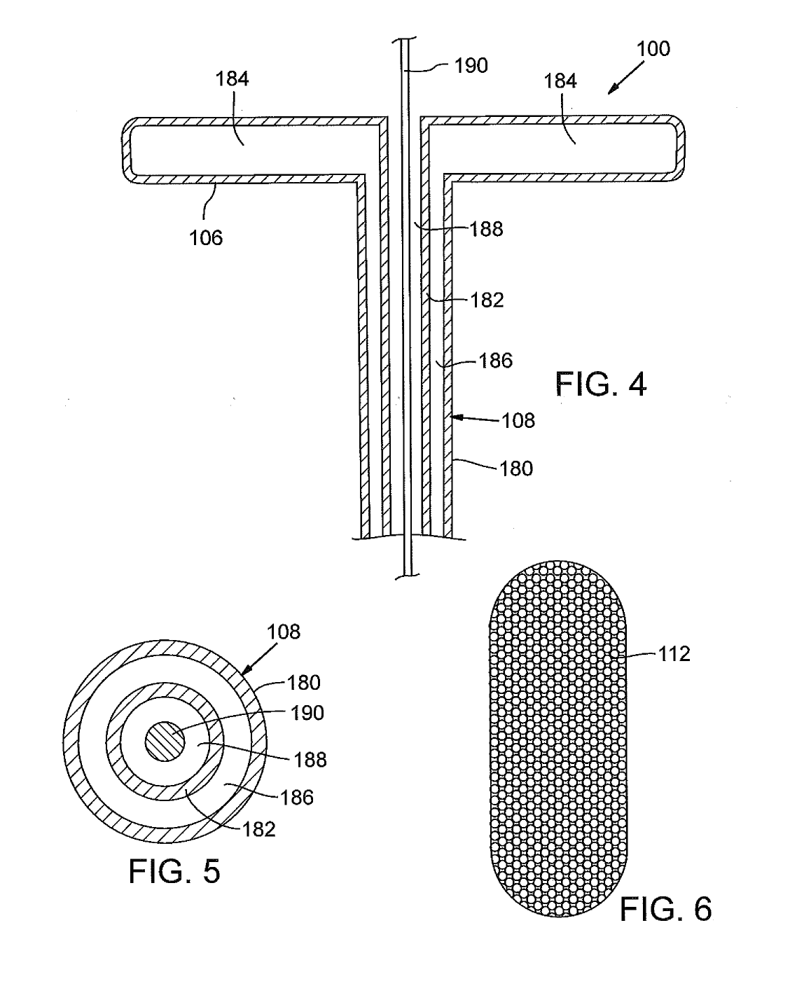

FIGS. 4-5 illustrate an embodiment of the embolic deflector 100 that is configured to be advanced over a guidewire. In the embodiment of FIGS. 4-5, the shaft 108 comprises an outer shaft 180 and an inner shaft 182 extending coaxially through the outer shaft 180. The proximal ends of the shafts 180, 182 can be connected to the handle 104 (FIG. 12). The flexible frame 106 comprises a lumen 184 extending throughout its structure (e.g., the struts 120, 122, 124). An inflation fluid lumen 186 is defined between the outer shaft 180 and the inner shaft 182. The distal end of the inflation fluid lumen 186 is in fluid communication with the lumen 184 of the frame 106 and the proximal end of the inflation fluid lumen 186 is in fluid communication with a source of inflation fluid. Hence, an inflation fluid source fluidly connected to the handle supplies an inflation fluid that can flow through the handle 104, the inflation fluid lumen 186, and into the lumen 184 to inflate the frame 106. The inflation fluid can also be withdrawn from the lumen 184 in order to deflate the frame 106, such as when it is to be removed from the body.

As also illustrated in FIGS. 4-5, the inner shaft 182 defines a guidewire lumen 188 extending along its length. As also illustrated, a guidewire 190 may be provided within the guidewire lumen 188 and in use, extends through the entire inner shaft 182 and the frame 106. The guidewire 190 can be introduced into a patient's vasculature prior to the deflector, after which the deflector 100 can be advanced over the guidewire 190, as further described below.

Additional lumen may be provided, depending upon the desired functionality of the embolic deflection system. For example, contrast dye or other flowable media may be introduced through a separate lumen extending through the outer shaft 180 or the inner shaft 182 to provide an elongate flow channel from the handle 104 to the distal opening. In addition or as an alternative to contrast dye, any of a variety of thrombolytic agents or other drugs identified elsewhere herein such as in the discussion of the membrane 112 may be infused. Normal saline, heparinized saline, or other rinse or flush media may also be introduced, such as to clear any adherent debris from the membrane 112. Alternatively, a separate lumen may be utilized to introduce any of a variety of additional structures, such as a pressure sensor to sense aortic blood pressure, or a cardiac output monitor to monitor blood flow or an emboli capture basket for positioning in the aorta downstream from the emboli deflector. Additional features may be added depending upon the desired functionality of the embolic deflection system.

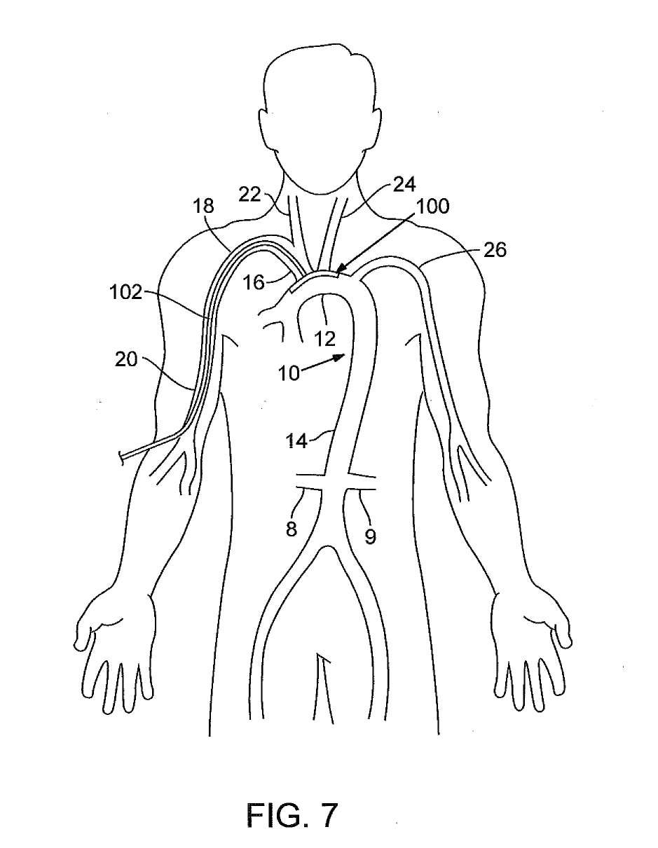

Referring now to FIG. 7, in one embodiment, a deflector 100 can be delivered via percutaneous or cut-down insertion into the right brachial artery 20, advanced to the right subclavian artery 18, and then guided into the aortic arch 12. The deflector 100 can then be deployed and inflated, and pulled back under traction into position to cover the ostia of the brachiocephalic artery 16 (which may also be referred to herein as the innominate artery or the brachiocephalic trunk) and left common carotid artery 24. The deflector 100 deflects emboli during cardiovascular procedures, allowing the flow of blood through deflector 100 and into the cerebral circulation (carotid arteries) sufficient to maintain perfusion to the brain and other vital structures, while at the same time not permitting the passage of emboli into the cerebrovascular circulation of a size which could cause stroke. Also illustrated in FIG. 7 for anatomical reference is the descending aorta 14, right common carotid artery 22, aorta 10, and left subclavian artery 26.

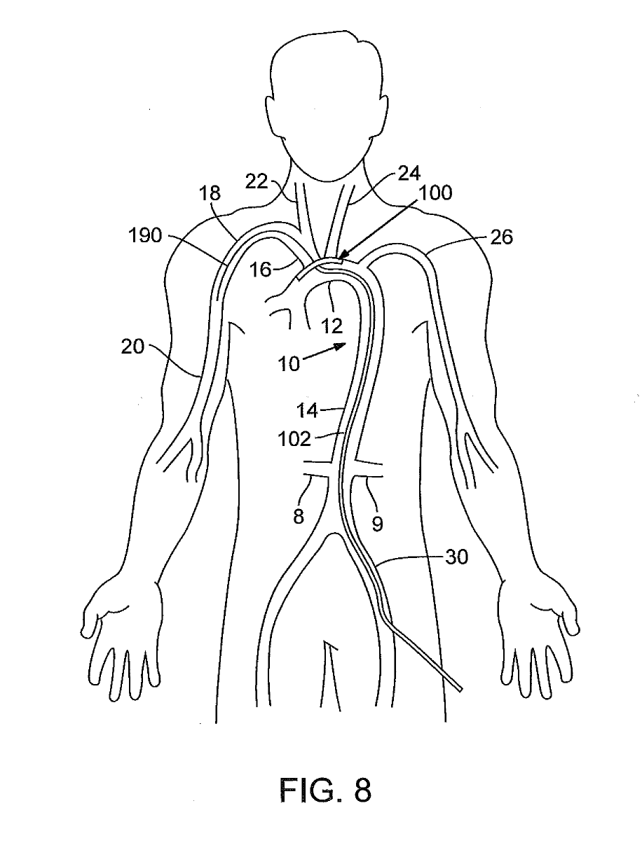

Referring now to FIG. 8, in one embodiment, the deflector 100 is delivered via percutaneous or cut-down insertion into a femoral artery (such as the left femoral artery 30) and is guided upstream from the descending aorta 14 into the aortic arch 12. After catheterization of the brachiocephalic artery 16, the device 100 is passed over a guidewire or through a lumen of a deployment catheter and brought into position and maintained under distal pressure covering the ostia of the brachiocephalic artery 16 and/or the left common carotid artery 24, and additionally the left subclavian artery 26 (not shown) in some embodiments.

Referring now to FIGS. 9A-9C, an exemplary procedure for percutaneous access to the circulation via an upper extremity (through any appropriate artery, such as the radial, ulnar, brachial, axillary, or subclavian artery) is depicted. Prior to inserting the deflector 100 into the patient's vasculature, a guidewire 190 can be advanced into the aortic arch 12 after exiting the innominate artery 16.

A separate delivery catheter 102 can then be advanced over the guidewire 190 to position a distal end of the delivery catheter 102 in or in the vicinity of the aorta. Additional details of the delivery catheter 102 and other mechanical components will be provided below. In general, the delivery catheter can comprise at least one central lumen for receiving the deflector 100 therethrough. The crossing profile of the system may be minimized by providing a delivery catheter 102 which comprises only a single lumen tube, such as a single lumen extrusion. This delivery tube may be advanced over the guidewire 190 into position within the aorta 10. The guidewire 190 can then be proximally retracted and removed from the delivery catheter 102, leaving the central lumen available to receive the deflection device 100 therethrough.

In the illustrated embodiment, the delivery catheter 102 is placed over the guidewire 190 and guided into the aortic arch 12. The guidewire 190 is retracted and the deflection device 100 is axially distally advanced through the central lumen thereby exposing the device 100 to the aortic arch 12 bloodstream (FIG. 9A). The device 100 is then inflated in the aortic arch 12 (FIG. 9B). A benefit is that the deflector 100 can be expanded or contracted within the aorta without contacting the wall on the inside radius of the thoracic aorta.

In alternative embodiments, the deflector 100 and the delivery sheath 102 can be advanced together as a single delivery apparatus 50 (FIG. 12) over the guidewire 190, with the distal end portion of the sheath 102 covering the frame 106, which is in a deflated configuration. The delivery apparatus 50 can be advanced until its distal end is located in the aorta. The frame 106 can be deployed from the sheath 102 by advancing the frame 106 distally from the sheath 102 or by retracting the sheath 102 relative to the frame 106. The frame 106 can then be inflated as shown in FIG. 9B.

In either case, once inflated, the device 100 is pulled back into position, covering the ostia 17 of the innominate artery 16 as well as the ostia 25 of the left common carotid artery 24 (FIG. 9C). The device 100 allows the passage of blood through to the carotid arteries 22, 24 while still deflecting emboli generated by aortic or cardiac surgery or other procedure away from these arteries, and downstream into the descending aorta. At the completion of the debris-producing concomitant procedure or following elapse of any other desired period of time, the device 100 can be deflated and withdrawn into the central lumen of deployment catheter 102 to completely encapsulate it prior to removal from the arm access artery (not shown).

In some embodiments, the deflector 100 can be retrieved into the sheath 102 by simply deflating the frame 106 and retracting the shaft 108 relative to the sheath 102. The flexible frame 106 folds together as the sheath forces the lateral ends of the lobes to be closed together. Once the deflector 100 is fully captured and changes into its collapsed configuration inside of the sheath 102, the sheath 102 and deflector 100 can then be removed from the body. Variations on the procedure could be employed to minimize intimal damage and/or potential for release of emboli during retrieval. One possible procedural variation would be for the user to advance the device and sheath tip into the aorta near the lesser curve of the arch, then re-sheath the device in that location.

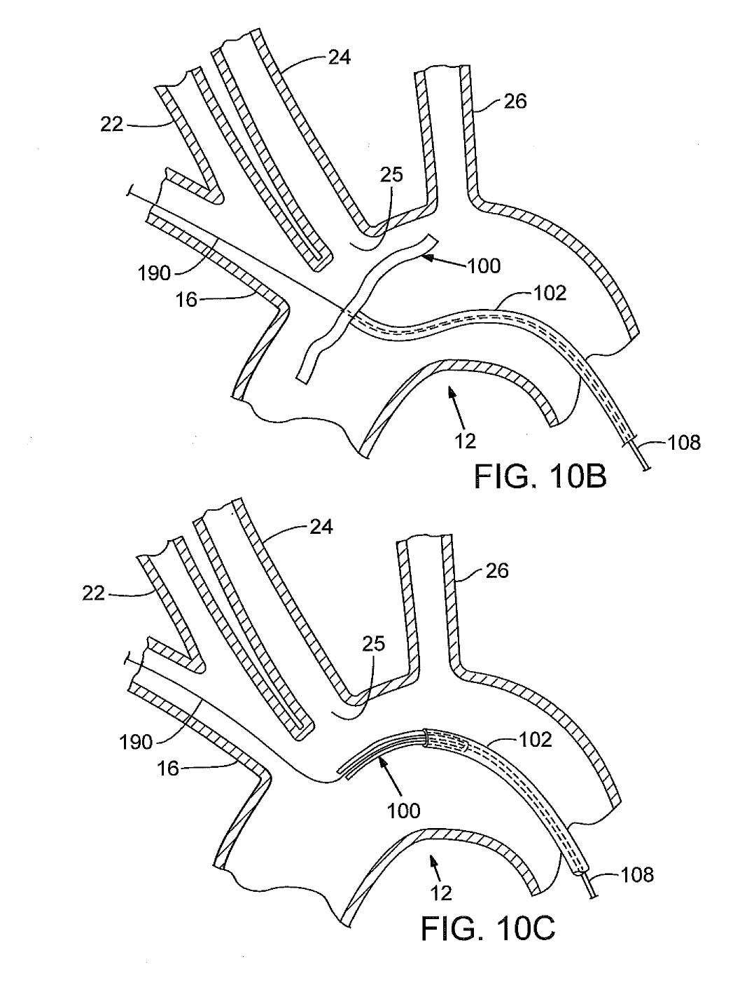

Referring now to FIGS. 10A-10C, in another embodiment, the innominate artery 16 is catheterized with a guidewire 190 placed via femoral access. Over the guidewire 190, the deflector deployment system is guided into position in the aortic arch 12, where the deflector 100 is deployed, for example, by retraction of the sheath 102. The sheath 102 can be advanced over the guidewire 190 prior to inserting the deflector 100 into the patient, or both can be inserted together and advanced over the guidewire 190 as a single apparatus. In either case, the frame 106 is inflated and then pushed, over the guidewire 190 in the innominate artery 16, into position securely covering the ostia 17 of the innominate artery 16 and ostia 25 of the left common carotid artery 24 (FIG. 10A). As discussed above, the deflector 100 allows the passage of blood through to the carotid arteries 22, 24, but deflects emboli generated by aortic or cardiac surgery away from these arteries. At the completion of the debris-producing concomitant procedure or other period of time elapsed, the frame 106 can be closed by deflating it (FIG. 10B). The frame 106 is then collapsed and withdrawn into a covering sheath 102 (FIG. 10C) to completely encapsulate it prior to removal from the leg access artery. Any trapped debris is enfolded within the collapsed frame 106, safely and securely within the covering sheath 102. The guidewire 190, the sheath 102, and the deflector 100 can then be withdrawn from the femoral access.

In still other embodiments in which the ostia of three side branch vessels, such as the brachiocephalic artery, left common carotid artery, and left subclavian arteries are all to be covered by a deflector 100, an alternative deployment method would be through insertion of the vessel into the left upper extremity, such as the left radial, ulnar, brachial, axillary, or subclavian arteries. The deflector 100 can be advanced into the aortic arch from the left subclavian artery, expanded, and then traction could be placed to create a seal with the aortic wall to cover the ostia of the three side branch vessels.

Since deployment of the embolic deflection device via a femoral artery access can require placement of the deployment catheter across the thoracic aorta, this approach may be desirable for use in conjunction with heart procedures accomplished surgically, transapically, or via alternate access pathways that do not involve traversing the thoracic aorta with the index procedure device.

In some embodiments, the device 100 could also be used with open or thoracoscopic cardiac or aortic procedures. In these cases, the device could be placed in either manner described above, or via direct puncture or via guidance under imaging, such as fluoroscopy, into the aorta, brachiocephalic artery, right or left subclavian artery, or other suitable vessel if the arch were exposed. If it were placed directly, it can be pushed into place as with the femoral approach. Alternatively, any appropriate surgical, percutaneous, or endoscopic procedure may be employed to place the device 100.

During deployment as described above, in an embodiment in which the deflector 100 is preloaded into the sheath 102 prior to advance to the treatment site, the deflector 100 may be locked in position relative to the sheath 102 using a rotating valve, torque control, or similar mechanism. The sheath 102 can then be held in position at the skin using, for example, a hemostat, clip, tape, Tegaderm™ or other adhesive film. The deflector 100 remains tethered by the shaft 108, and tensioned against the vessel wall by application of tractional force external to the patient. In some embodiments, a deployment system includes an intermediate biasing structure that reversibly locks the deflector 100 in position when a predetermined amount of tractional force is applied by a physician to place the deflector 100 in sealing contact against the vessel wall. The intermediate biasing structure could be, for example, a spring having a predetermined spring bias. Such an intermediate biasing structure could be advantageous in eliminating potential variability from physician in the amount of tractional force applied, to create an optimal seal as well as a safety feature to avoid damage to the intimal vessel wall or other structures. The deflector 100 and/or shaft 108 may be elastic to accommodate movement or shifting during use, so as to maintain protection of the vasculature. The deflector 100 is preferably tethered to permit repositioning or removal at any time.

In some embodiments, the method can be modified to account for patient anatomical abnormalities, such as abnormalities of the aortic arch. In some embodiments, the deflector 100 can be sized to cover the ostia of a single vessel, or a first deflector 100 can be sized to cover the ostia of a first vessel, and a second deflector 100 can be sized to cover the ostia of a second vessel. For example, some patients may have an aortic arch side branch vessel abnormality where the right common carotid artery and the left common carotid artery are both direct side branch vessels off the aortic arch, or the right and left common carotid arteries bifurcate off a single side branch vessel off the aortic arch. The patient's vascular anatomy can be first determined, such as by angiography, CT angiography, MRI, Doppler ultrasound, or other method. One, two, or more deflecting devices 100 can be positioned at or near the ostia of one, two, three, or more side branch vessels (potentially more in patients with a double aortic arch) such that the end result is that all emboli larger than a predetermined size are prevented from reaching the brain including brainstem, eyes, or other critical structures perfused by the carotid and/or vertebral arteries.

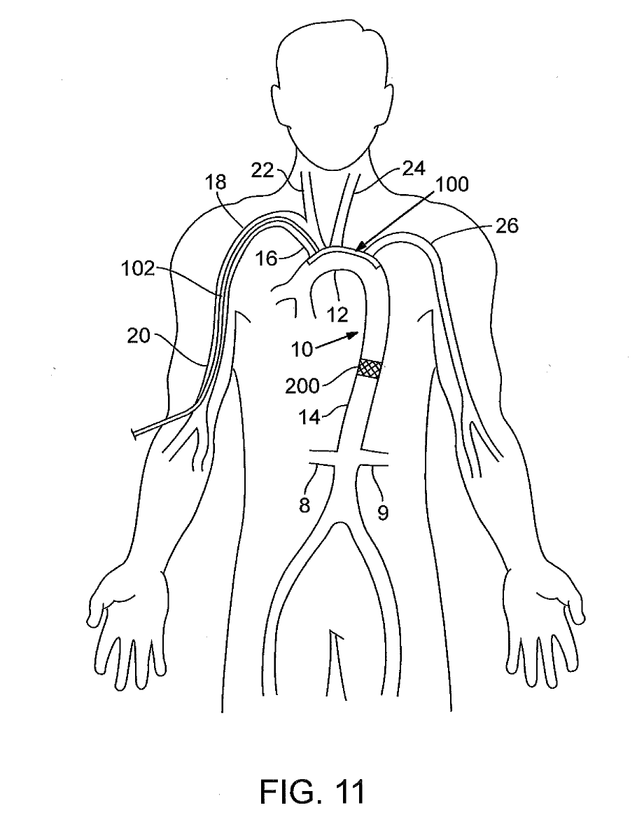

In addition to deflectors 100 as described herein, conventional embolic protection devices including arterial and venous filters can also be sized and configured to be placed in a main vessel over the ostia of at least a first, second, or more side branch vessels and used with the methods disclosed herein, such as, for example, the brachiocephalic artery and the left common carotid artery as described above. In some embodiments, an embolic protection device 100 sized and configured to span the aorta, such as the descending aorta, can be placed downstream of the deflector in the aortic arch to capture emboli before reaching the ostia of the renal arteries. FIG. 11 schematically illustrates a deployed deflector 100 that can cover the ostia of the brachiocephalic 16, left common carotid 24, and also the left subclavian artery 26. An adjunct embolic filter 200 can be placed in the aorta 10 downstream of the ostia of the left subclavian artery 26 but upstream of the ostia of the left 9 and right 8 renal arteries in order to trap emboli prior to potential embolization into the renal arteries 8, 9. In some embodiments, the embolic filter 200 can be a stand-alone filter as shown temporarily positioned and secured in the aorta via any desired mechanism, such as with anchors such as barbs, attached to a control line extending from the left or right femoral arteries or a right or left upper extremity artery, or tethered to the deflector 100 in some embodiments. The embolic filter 200 can then be removed from body following completion of the index procedure. Some examples of embolic protection devices 100 including filters that can be used or modified for use with the methods described herein, as well as deployment and removal methods for those filters can be found, for example, in U.S. Pat. No. 4,619,246 to Molgaard-Nielsen et al., U.S. Pat. No. 5,634,942 to Chevillon et al., U.S. Pat. No. 5,911,734 to Tsugita et al., U.S. Pat. No. 6,152,946 to Broome et al., U.S. Pat. No. 6,251,122 to Tsukernik, U.S. Pat. No. 6,346,116 to Brooks et al., U.S. Pat. No. 6,361,545 to Macoviak et al., U.S. Pat. No. 6,375,670 to Greenhalgh, and U.S. Pat. No. 6,447,530 to Ostrovsky et al., all of which are hereby incorporated herein by reference in their entireties.

FIG. 13 shows an embodiment of a torque control mechanism 300, which functions similar to that of a wire pin vise, can be used to stabilize the deflector 100 (not shown) during packaging, and also as a proximal handle to help grip and manipulate the shaft 108 during use. Transmission of torque from the shaft 108 to the frame 106 can be particularly advantageous while manipulating the deflector 100 within the vasculature, in order to rotate a radially asymmetric deflector 100 into its desired location, such as to cover the ostia of the brachiocephalic artery and the left common carotid artery, for example. In some embodiments, the torque control mechanism 300 can be used to grip guidewires up to 0.038″ in diameter and employs a clamp 302 that can be rotated in an appropriate direction by an operator to reversibly lock and unlock onto the shaft 108. The torque control mechanism 300 can be integrated in the handle 104 (FIG. 12) of the delivery apparatus or it can be a separate component located adjacent the handle 104 at the proximal end of the delivery apparatus.

The torque transmission capability of the shaft 108 will generally decline as the shaft 108 is made longer. Torque transmission capabilities of the shaft 108 may be enhanced by constructing the shaft 108 of non-polymeric material (e.g. metal tube or hypotube). Alternatively, shaft 108 may be fabricated such as by wrapping a first polymeric filament helically around a mandrel in a first direction, and bonding a second polymeric filament wrapped helically in a second, opposing direction around the first wrapping. Additional layers of helical wrapping or braided constructions can provide relatively high torque transmission, as is understood, for example, in the intracranial microcatheter arts.

As illustrated in FIG. 14, the device 100 can be loaded through a loader 420, which can be operably connected to, in some embodiments, a blunt-tipped introducer sheath 400 to facilitate insertion of the delivery apparatus into the patient's vasculature. The introducer 400 can be, for example, 6 French in size, and can allow the deflecting device 100 to be flushed and back-loaded. The introducer 400 includes a hemostasis valve within a housing 402 and an introducer shaft 404 extending from the housing 402. The housing 402 can have a flush port 406 (with stopcock) and a length of tubing 408, which can be optionally attached. The deflector 100 can be initially collapsed into the loader 420 to evacuate all air. The deflector 100 then passes through the hemostasis valve within the housing 402, the shaft 404, and the delivery sheath 102. Alternatively, the deflector 100 and the sheath 102 can be inserted together through the loader 420 and the introducer sheath 400.

In addition to the introducer 400 described above, FIG. 14 illustrates one, two, or more other components of a deflector system or kit including a deployment system 500 that can be packaged together in a sterile fashion, and ready for physician use. The system 500 also includes the deflector 100 as disclosed elsewhere herein, sheath 102 housing the shaft 108 (not shown) of the deflector 100, torque control 300 housing a length of guidewire 190, and other loading tools (not shown) as required.

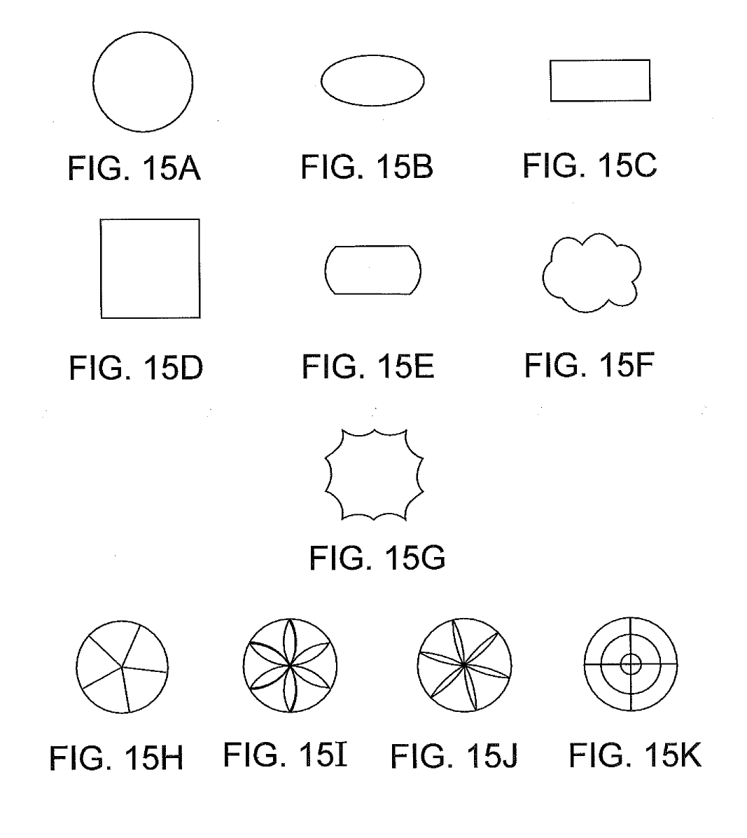

FIGS. 15A-15K are end schematic views of various configurations of alternative deflector frames, according to some embodiments of the invention. For example, in FIG. 15A, the deflector frame 106 is radially symmetric and dome-shaped like an umbrella. The edge of the umbrella can be a flexible, donut-shaped element, similar to the edge of a diaphragm, allowing a good seal with the curved aortic wall. A ring can define the edge in some embodiments. The dome part of the umbrella can include inflatable struts to assist in the opening and closing of the umbrella and to help maintain its position. The center of the frame 106 can have an inflatable hub on the inside surface to which the struts are connected. The device can be pushed out of the delivery catheter with a tube, wire or other member that engages this hub. The hub can remain attached to the deflector shaft 108, and the guidewire 190 can be used to pull the deflector 100 into position. The porous membrane 112 is attached, in some embodiments, at the highest point of the profile to assist with an umbrella-like deflection of clot or debris. The device 100 may be provided with radiopaque markers 170 or metal parts which are radiopaque as described elsewhere in the application.

Further embodiments of top views of deflector frames 106 illustrated include oval (FIG. 15B), rectangular (FIG. 15C), square (FIG. 15D), rectangular with rounded lateral ends (FIG. 15E), cloud-shaped (FIG. 15F), starburst-shaped (FIG. 15G). FIGS. 15H-15K illustrate phantom plan views illustrating frames with 5 struts and a central hub (FIG. 15H), having wide (FIG. 15I) and narrow (FIG. 15J) petals, or with concentric elements (FIG. 15K). Other embodiments of the deflector frame have a rolled edge, or a flat porous edge. Another embodiment of the device 100 has anchors such as barbs, along the frame 106, e.g., at the lateral edges which help to maintain its position during the procedure.

Another embodiment of the deflector 100 is parachute-like, with annular gasket at its edge, which can be an inflatable component. The gasket can be held firmly in position over the ostia of the appropriate vessels, such as the brachiocephalic and left common carotid arteries. A billowy porous middle section can deflect or trap embolic debris on its exterior surface while causing minimal resistance in the aorta. A middle portion can be inverted as it is removed by pulling on wires attached to its center, capturing any clot stuck to it. Alternatively, the center of the device can be a screen, which fits more snugly against the aortic wall, with a very small profile, further preventing resistance. Again the device 100 can be removed by inversion, capturing any emboli or thrombus that may accumulate on the membrane 112 or other component of the deflector prior to removal.

Another embodiment of the deflecting device 100 includes a rib-supported or self-supporting inflatable spherical frame 106 covered by porous membrane 112, which may be distorted into a flat or semi-flat shape for covering one, two, or more vessel ostia by withdrawing a wire attached to one side of the sphere. The device 100 may be oval, rectangular or of another shape, some of which are illustrated above, to assist in sealing of the edge against the wall of the aorta, covering the ostia of, for example, both the brachiocephalic and left common carotid arteries and maintaining a low profile within the lumen of the aorta.

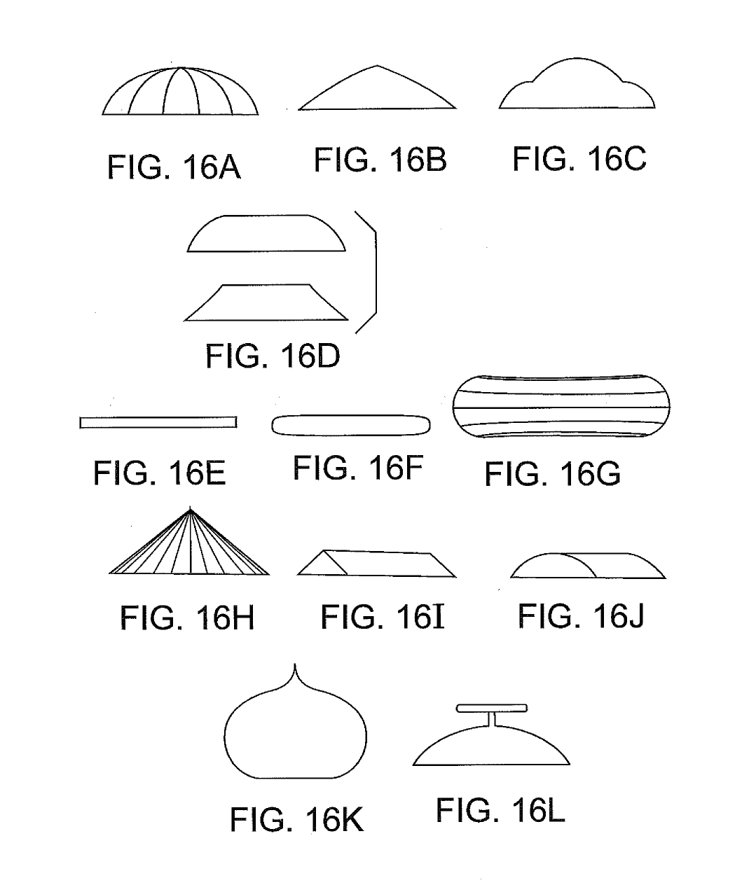

Side view depth profiles of deflector frames 106 are illustrated in FIGS. 16A-16L. These depth profiles include onion-shaped (FIG. 16A), frustoconical (FIG. 16B), bi-level with multiple curvatures (FIG. 16C), bilevel concave or convex (FIG. 16D), flat (FIG. 16E), slightly rounded (FIG. 16F), oval (FIG. 16G), pyramidal (FIG. 16H), tent-shaped and pointed (FIG. 16I) or more rounded (FIG. 16J), tear-drop shaped (FIG. 16K), or conical with a projection that may extend to the opposite wall of the aortic lumen, such as for improved anchoring (FIG. 16L). The deflector can include 1, 2, 3, or more frame and/or membrane layers and may be comprised of overlapping or connecting components.

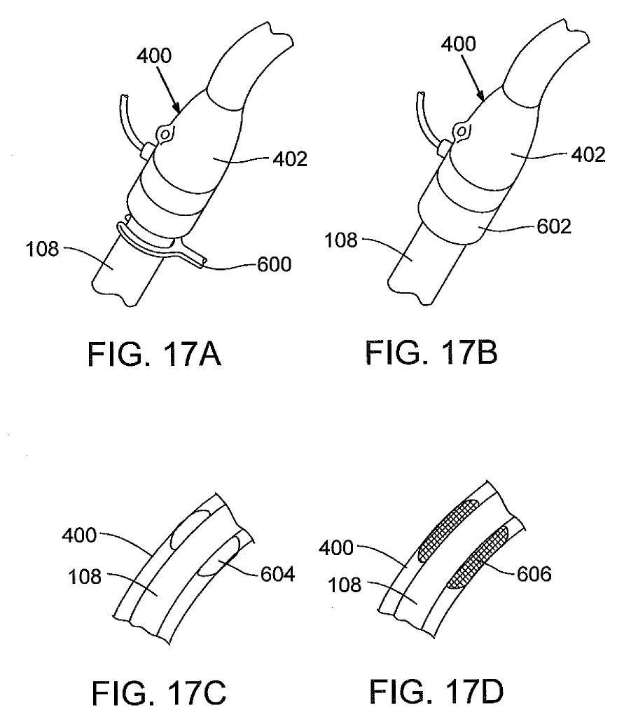

FIGS. 17A-17D illustrate different embodiments of external locking mechanisms that can assist in maintaining the deflector 100 in a desired position in the body. FIG. 17A illustrates a clamp 600 that can fix the shaft 108 of the deflector 100 relative to the introducer sheath 400. FIG. 17B illustrates a threaded twist screw 602 functioning similarly to that of the clamp 600 of FIG. 17A. FIGS. 17C-17D illustrate an expandable member configured to reside within the introducer sheath 400 and at least partially surround the shaft 108 of the deflector 100 to prevent proximal or distal movement of the shaft 108 within the introducer sheath 400. An inflatable balloon 604 is illustrated in FIG. 17C, that can be inflated or deflated, for example, via a separate inflation media lumen within the introducer sheath 400. A stent-like sleeve 606 is illustrated in FIG. 17D. In some embodiments, the sleeve 606 can have shape memory properties and can radially expand or contract with the application of heat or cold to the sleeve 606. In some embodiments, the locking mechanism can be incorporated with the torque control mechanism 300 as previously described. In addition, in the embodiments of FIGS. 17A-17D, components 600, 602, 604, 606 can be used to fix the sheath 102 relative to the introducer sheath 400 where the delivery apparatus comprises a sheath 102 extending over the shaft 108 and is configured to selectively lock the position of the shaft 108 relative to the sheath 102.

Other delivery techniques and configurations for deflectors are described in U.S. Patent Publication No. 2010/0179585, which is incorporated herein by reference.

The embodiments described herein and illustrated in the figures provide several advantages over the prior art, including the ability to create deflectors 100 of shapes and sizes not possible or practical by other techniques. The risk of injury due to a metal-framed device “sweeping” across the surface of a patient's circulation is also reduced. Other potential advantages are cost and time to manufacture, as well as a reduced profile and required introducer size (smaller being better).

For purposes of this description, certain aspects, advantages, and novel features of the embodiments of this disclosure are described herein. The disclosed methods, devices, and systems should not be construed as limiting in any way. Instead, the present disclosure is directed toward all novel and nonobvious features and aspects of the various disclosed embodiments, alone and in various combinations and sub-combinations with one another. The methods, devices, and systems are not limited to any specific aspect or feature or combination thereof, nor do the disclosed embodiments require that any one or more specific advantages be present or problems be solved.

Although the operations of some of the disclosed methods are described in a particular, sequential order for convenient presentation, it should be understood that this manner of description encompasses rearrangement, unless a particular ordering is required by specific language. For example, operations described sequentially may in some cases be rearranged or performed concurrently. Moreover, for the sake of simplicity, the attached figures may not show the various ways in which the disclosed methods can be used in conjunction with other methods.

As used herein, the terms “a”, “an” and “at least one” encompass one or more of the specified element. That is, if two of a particular element are present, one of these elements is also present and thus “an” element is present. The terms “a plurality of and “plural” mean two or more of the specified element.

As used herein, the term “and/or” used between the last two of a list of elements means any one or more of the listed elements. For example, the phrase “A, B, and/or C” means “A,” “B,” “C,” “A and B,” “A and C,” “B and C” or “A, B and C.”

As used herein, the term “coupled” generally means physically coupled or linked and does not exclude the presence of intermediate elements between the coupled items absent specific contrary language.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. I therefore claim as my invention all that comes within the scope and spirit of these claims

I claim:

1. An inflatable embolic deflector comprising:
an elongate shaft having a proximal end and a distal end, and including an inflation fluid lumen extending therethrough;
a flexible inflatable frame, defining at least one opening, coupled to the distal end of the elongate shaft, the flexible inflatable frame comprising a lumen in fluid communication with the inflation fluid lumen, the flexible inflatable frame being configured to inflate when a pressurized inflation fluid is introduced through the inflation fluid lumen and into the lumen of the flexible inflatable frame; and
a membrane spanning the at least one opening, the membrane having a permeability such that blood can pass through the membrane but emboli greater than a predetermined size cannot pass through the membrane.

2. The deflector of claim 1, wherein the shaft comprises an outer shaft and an inner shaft extending through the outer shaft, the inner shaft defining a guidewire lumen for receiving a guidewire.

3. The deflector of claim 2, further comprising a guidewire extending through the guidewire lumen of the shaft.

4. The deflector of claim 1, wherein the flexible inflatable frame comprises at least one radiopaque marker.

5. The deflector of claim 1, wherein the flexible inflatable frame defines a plurality of openings and a membrane is disposed in each opening.

6. The deflector of claim 1, wherein the flexible inflatable frame comprises:
first and second inflatable side struts; and
first and second inflatable end struts extending between respective ends of the side struts;
wherein the at least one opening is defined between the side struts and the end struts.

7. The deflector of claim 6, wherein:
the flexible inflatable frame further comprises an inflatable central strut extending between central portions of the side struts;
a first opening is defined between the first inflatable side strut, inflatable central strut, and the side struts;
a second opening is defined between the second inflatable side strut, inflatable central strut, and the side struts; and
the first and second opening have respective first and second membranes disposed therein.

8. The deflector of claim 7, wherein the elongate shaft is coupled to the flexible inflatable frame at a central portion of the inflatable central strut.

9. The deflector of claim 1, further comprising an elongated delivery sheath extending coaxially over the elongate shaft.

10. The deflector of claim 1, further comprising a handle coupled to the proximal end of the elongate shaft and a source of an inflation fluid fluidly coupled to the handle, the handle configured to receive an inflation fluid from the source and introduce the inflation fluid into the inflation fluid lumen of the elongate shaft for inflating the flexible inflatable frame.

11. The deflector of claim 1, wherein the flexible inflatable frame has a concave shape when inflated.

12. A method for deflecting emboli within a patient's vasculature comprising:
inserting an inflatable embolic deflector into the patient's vasculature, the inflatable embolic deflector comprising a shaft, an inflatable frame coupled to a distal end of the shaft, and a membrane supported by the inflatable frame, the membrane having a permeability such that blood can pass through the membrane but emboli greater than a predetermined size cannot pass through the membrane, wherein the inflatable frame is in a deflated configuration when introduced into the patient's vasculature;
inflating the inflatable frame within the patient's vasculature; and
situating the inflated inflatable frame to cover an ostium of a lumen of the patient's vasculature such that that blood can pass through the membrane and into the lumen but emboli greater than the predetermined size cannot.

13. The method of claim 12, wherein the ostium of the lumen comprises one of:
the ostium of the patient's brachiocephalic artery; and
the ostium of the patient's left common carotid artery.

14. The method of claim 12, wherein the act of inserting is performed through the patient's right brachial artery.

15. The method of claim 14, wherein the act of situating is performed by retracting the inflatable frame proximally.

16. The method of claim 12, wherein the act of inserting is performed through one of the patient's femoral arteries.

17. The method of claim 16, wherein the act of situating is performed by extending the inflatable frame distally.

18. The method of claim 12, further comprising implanting a prosthetic valve in the patient's heart while the inflatable embolic deflector is positioned to cover the ostium.

19. The method of claim 12, further comprising performing angioplasty or valvuloplasty while the inflatable embolic deflector is positioned to cover the ostium.

20. The method of claim 12, wherein the act of situating the inflated inflatable frame to cover an ostium comprises situating the inflated inflatable frame to cover two or more ostia.
